(19) United States
(12) Patent Application Publication
(10) Pub. No.: US 2022/0298332 A1
(43) Pub. Date: Sep. 22, 2022

(54) NATURAL RUBBER LATEX SPONGE PREVENTED FROM DISCOLORATION AND PROCESS OF PRODUCING THE SAME

(71) Applicant: Yukigaya Chemical Industry Co., Ltd., TOKYO (JP)

(72) Inventors: Hitoshi HONDA, Inashiki-shi (JP); Kazuo OBATA, Inashiki-shi (JP); Megumi FURUWATARI, Inashiki-shi (JP); Ai HASEGAWA, Inashiki-shi (JP)

(21) Appl. No.: 17/655,591

(22) Filed: Mar. 21, 2022

(30) Foreign Application Priority Data
Mar. 22, 2021 (JP) 2021-047616

(51) Int. Cl. C08L 7/02; C08J 9/14; C08J 9/12; C12P 5/00; B08B 3/08; A45D 34/04

(52) U.S. Cl. CPC C08L 7/02; C08J 9/14; C08J 9/127; C12P 5/007; B08B 3/08; A45D 34/04; C08J 2307/02; A45D 2200/1009

(57) ABSTRACT

To provide natural rubber latex sponge with little pinking, and in particular deproteinized natural rubber latex sponge with low allergic properties. Natural rubber latex sponge is discolored when irradiated with light for 24 hours in an ammonia atmosphere of 0.03 g/l. The discoloration of the natural rubber latex sponge causes a color difference ΔE*ab of 0 to 10 between before and after the irradiation. The natural rubber latex sponge is produced from deproteinized natural rubber latex or latex obtained by causing a protein decomposition enzyme to react with natural rubber latex to decompose proteins.

### CROSS REFERENCE TO RELATED APPLICATION

This claims priority from Japanese patent application no. 2021-047616, filed Mar. 22, 2021, incorporated herein by reference.

### TECHNICAL FIELD

The present invention relates to a process of producing natural rubber latex sponge prevented from discoloration over time, and to natural rubber latex sponge that is produced by the process and that is not easily discolored over time.

### BACKGROUND

Natural rubber latex sponge is mainly made from latex obtained by making a cut on the trunk of a Para rubber tree (Hevea brasiliensis) which is an evergreen tree. The latex is refined, condensed, and combined with a vulcanizing agent, a vulcanization accelerator, a frothing agent, a foam stabilizer, zinc oxide, a gelling agent, etc. to obtain a raw material to be foamed. The raw material is mixed with air to be frothed, and subjected to gelation molding. The resulting material is heated and vulcanized to impart rubber elasticity, completing the natural rubber latex sponge. The chemical makeup of the natural rubber is polyisoprene. The polyisoprene produced from the Para rubber tree has a cis steric structure, and has properties such as high strength and low modulus compared to synthetic polyisoprene. Sponge with high elasticity and that is soft and has a good touch to the skin can be produced by making use of such properties, and is used as cosmetic sponge etc.

The natural rubber latex sponge is produced with both use of a small amount of crude oil and emission of a small amount of carbon dioxide, compared to synthetic rubber sponge which is produced from crude oil as a raw material. The natural rubber latex sponge matches the SDGs in many respects, and is friendly to the global environment.

While the natural rubber latex sponge has the advantages described above, the natural rubber latex sponge is known to be subjected to pinking in which the natural rubber latex sponge is discolored to assume a red to reddish brown color over a period of three months to half a year, although to different degrees. Therefore, the sponge is colored in advance so that the discoloration will be unnoticeable. The sponge is packed in a light-shielding hermetic wrapping material etc. not to be discolored before the time of use.

The pinking is induced by light and air, and the sponge is not discolored if the sponge is stored away from light and air. The sponge is simply discolored and does not become brittle or hardened because of light and heat, unlike oxidation degradation which is promoted by light and heat. The pinking is not improved by adding an antioxidant. The pinking is a phenomenon that is peculiar to the natural rubber latex sponge. From the above, the prevent inventors consider that the discoloration is caused by polyphenols or lipids other than the resins contained in the natural rubber latex.

The natural rubber is known to cause latex allergies due to protein contained in the latex, and use of the natural rubber with direct contact with the skin is avoided. On the other hand, deproteinized natural rubber latex with reduced allergic properties has been produced. Sponge made from the deproteinized natural rubber latex as a raw material is preferable in double meanings. That is, the sponge is hypoallergenic and can be used with security, and has low modulus and is flexible. However, the sponge is subjected to the pinking discussed earlier to a greater degree than that caused in sponge produced from latex before removal of the proteins, and use of the sponge made from the deproteinized natural rubber latex is still limited.

Patent Document 1, JP 6-240003 A describes pinking of finger stalls made from natural rubber latex. The document indicates that the finger stalls are discolored by ammonia and light, and that the finger stalls are not discolored if the finger stalls are wrapped together with a substance that absorbs ammonia.

Patent Document 2, JP 10-139926 A describes a process of producing latex from natural rubber latex from which proteins have been removed. The document indicates that deproteinized natural rubber latex is degraded during storage, that a phenolic antiaging agent (antioxidant) is added in order to prevent such degradation, and that the deproteinized natural rubber latex is discolored by the addition of the agent during storage.

Patent Document 3, JP 7-109380 A describes discoloration of a phenolic antioxidant. The document indicates that a carbonyl compound having a conjugated double bond such as a quinone structure or a stilbenequinone structure, generated as a result of oxidation of an alkyl-substituted phenolic antioxidant such as BHT (2,6-di-tertiary butyl-p-cresol), has a significantly large absorption constant in the visible region and therefore is discolored even if generated in a minute amount, and that the discoloration of the carbonyl compound is reduced by combining a specific amide compound therewith.

### DISCLOSURE OF THE INVENTION

### Technical Problem

While the technique according to Patent Document 1, JP 6-240003 relates to a measure against the pinking, however, the problem is not fundamentally solved. While the technique according to Patent Document 2, JP 10-139926 A relates to the production of deproteinized natural rubber latex, sponge prepared by the technique is subjected to the pinking to a greater degree than sponge produced from latex before a deproteinization treatment. While the technique according to Patent Document 3, JP 7-109380 A is intended to prevent degradation due to heat and light by adding a phenolic antioxidant, the technique does not improve the pinking.

In order to address the foregoing issue, it is an object of the present invention to provide natural rubber latex sponge, in particular deproteinized natural rubber latex sponge with low allergic properties, to be subjected to pinking to a small degree.

### Means for Solving the Problem

The invention provides a natural rubber latex sponge which is not easily discolored over time, characterized by comprising foam-molding natural rubber latex, and cleaning the natural rubber latex using an oxidizing cleaning liquid.

In addition, the invention provides natural rubber latex sponge that is discolored when irradiated with light for 24 hours in an ammonia atmosphere of 0.03 g/l, discoloration of the natural rubber latex sponge causing a color difference of 0 to 10 between before and after the irradiation in terms of a color difference (ΔE*ab) in an L*a*b* color system prescribed by JIS Z 8730. The color difference (ΔE*ab) is calculated in accordance with an opponent color space CIE 1976 (L*a*b* color space), abbreviated to CIE Lab, determined by the International Commission on Illumination, and also determined by ISO 7724 and ASTM D2244.

In addition, the invention provides the natural rubber latex sponge, in which the natural rubber latex sponge is produced from deproteinized natural rubber latex.

In addition, the invention provides a process of producing the natural rubber latex sponge, including causing a protein decomposition enzyme to act on natural rubber latex, and foam-molding the natural rubber latex with decomposed matter remaining.

In addition, the invention provides a process of producing the natural rubber latex sponge, including foam-molding natural rubber latex, and cleaning the natural rubber latex using a cleaning liquid of sodium percarbonate.

### Advantageous Effects of the Invention

The natural rubber latex sponge according to the present invention can provide sponge that has a uniform and fine cell structure and that is in a light color and is resistant to discoloration over time. Consequently, the degree of freedom in merchandise planning can be increased to finely meet the needs of the customers. Since discoloration is not caused over time, no much attention is required to manage the inventory period in merchandise management.

Sponge from which proteins have been removed can be provided by using deproteinized natural rubber latex, and can be used with security with no worries about allergies. At the same time, the sponge can have low modulus and a good touch to the skin. Further, foam molding is performed after the proteins in the natural rubber latex are decomposed. Thus, it is possible to provide sponge that has low modulus and a good touch to the skin, with no worries about allergies without increasing the cost, as the latex cleaning and refining steps can be omitted.

### DESCRIPTION OF EMBODIMENTS

First Embodiment—Natural rubber latex sponge according to a first embodiment of the present invention will be described.

The natural rubber latex sponge according to the present invention is produced by foam-molding a foam latex composition and cleaning the molded composition using an oxidizing cleaning liquid.

The foam latex composition is prepared by mixing natural rubber latex with compounding agents such as a vulcanizing agent, a vulcanization accelerator, an antioxidant, a frothing agent, a foam stabilizer, zinc oxide, and a gelling agent.

As the natural rubber latex, natural rubber latex prepared by processing sap collected from Para rubber trees may be used. Specific examples include natural rubber latex of a high ammonia type, a low ammonia type, and an alkali added type, and deproteinized natural rubber latex with a reduced protein content. The natural rubber latex is preferably used with the pH thereof adjusted to 9.0 to 10.0. The concentration of the solid content of the natural rubber latex may be 55 to 65%.

As the deproteinized natural rubber latex, deproteinized natural rubber latex prepared by removing a protein content from the natural rubber latex may be used. The natural rubber latex contains about 1.5% of a protein component, and is known to contain 15 kinds of allergen components (Hey b 1 to 15). Among these, Hey b 1, Hey b 3, Hey b 5, and Hey b 6.02 are considered to be major allergens that sensitize many people. The allergen proteins can be measured by the ELISA method, which detects the allergen proteins using monoclonal antibodies, and Hey b 6.02 can be detected with particularly high sensitivity. The deproteinized natural rubber latex according to the present invention may contain the Hey b 6.02 protein in an amount of 0.004 to 0.15 μg per gram of the solid content of the latex. The Hey b 6.02 protein content may be preferably 0.008 to 0.050 μg/g, more preferably 0.008 to 0.025 μg/g.

The deproteinized natural rubber latex is prepared by causing a protein decomposition enzyme to act on the natural rubber latex to decompose proteins bound to rubber molecules and render the natural rubber latex soluble in water, and thereafter removing the proteins and the decomposed matter through cleaning in which the natural rubber latex is diluted with water and subjected to condensation through centrifugal separation several times repeatedly. While specific examples include Selatex (registered trademark) available from Sumitomo Rubber Industries, Ltd., the present invention is not limited thereto.

It is also possible to cause a protein decomposition enzyme to act on the natural rubber latex to decompose proteins so as to cause no allergic reactions, and perform foam molding with the decomposed matter remaining. In this case, an alkaline protease is preferably used as the protein decomposition enzyme, and can be added to the latex as an aqueous solution or a suspension of about 10%. The protein decomposition enzyme may be used in an amount of 0.01 to 0.5 parts by weight, preferably 0.05 to 0.2 parts by weight, per 100 parts by weight of the solid content of the latex. If the amount of the protein decomposition enzyme is less than that, the proteins are not decomposed sufficiently, and the proteins remain. If the amount of the protein decomposition enzyme is more than that, a rough foaming failure may be caused during foam molding to be discussed later. The protein decomposition enzyme preferably adjusts the pH of the latex to 9.0 to 10.0 and causes an alkaline protease to act on the latex, which facilitates foam molding and improves the protein decomposition efficiency.

The latex to which the enzyme has been added is heated to 40 to 60° C. to promote a reaction to decompose the proteins, and thereafter kept warm or slowly cooled to allow the reaction to proceed. The enzyme treatment may take one to 24 hours.

The proteins act to stabilize the latex, and are rendered unstable by the enzyme treatment. Therefore, a surface active agent is preferably added to the latex in advance. A frothing agent to be discussed later is preferably used as the surface active agent.

When foam molding is performed using the latex with the proteins decomposed using the protein decomposition enzyme and with the decomposed matter remaining, foaming is performed well, and sponge with fine and uniform cells can be molded. The present inventors consider that this is because the decomposed matter from the proteins improves foaming properties.

As the latex, natural rubber latex and deproteinized natural rubber latex may be used, and various kinds of synthetic rubber latex and emulsion may be used in combination therewith. Examples include synthetic rubber latex such as butadiene rubber (BR), styrene-butadiene rubber (SBR), acrylonitrile-butadiene rubber (NBR), chloroprene rubber (CR), and isoprene rubber (IR), and also include synthetic rubber latex prepared by copolymerizing such synthetic rubber latex with a polymerizable monomer such as an acrylic ester and a methacrylic ester. As the emulsion, emulsion of synthetic resins may be used, and various kinds of anionic emulsion such as acrylic emulsion and urethane emulsion may be used.

The vulcanizing agent and the vulcanization accelerator are combined with the latex to impart rubber elasticity to the latex sponge according to the present invention. The vulcanizing agent may be sulfur, sulfur donor, zinc oxide, etc. The vulcanizing agent cross-links rubber molecules or rubber particles to impart rubber elasticity. The vulcanization accelerator activates the vulcanizing agent, and helps the vulcanizing agent cross-link rubber molecules or rubber particles in a shorter time and at a lower temperature to impart rubber elasticity. The vulcanization accelerator may be a thiazole vulcanization accelerator such as MZ (zinc salt of 2-mercaptobenzothiazole), a dithiocarbamic acid vulcanization accelerator such as EZ (zinc diethyldithiocarbamate) and BZ (zinc dibutyldithiocarbamate), a thiourea vulcanization accelerator such as EUR (N,N′-diethylthiourea), a guanidine vulcanization accelerator, a xanthogenic acid vulcanization accelerator, a thiuram vulcanization accelerator, etc. These may be used singly or in combination thereof.

The amounts of the vulcanizing agent and the vulcanization accelerator to be used differ more or less depending on the latex to be used. However, 0.5 parts by weight to 3.0 parts by weight of the vulcanizing agent may be used, and 0.7 parts by weight to 4.0 parts by weight of the vulcanization accelerator may be used, per 100 parts by weight of the latex.

The antioxidant prevents oxidation degradation of the latex sponge according to the present invention, and may be a phenolic antioxidant. Examples of the phenolic antioxidant include a bisphenol antioxidant such as BHT (2,6-di-tert-butyl-p-cresol) and Sumilizer BBM (registered trademark) and a polyphenol antioxidant such as Irganox 1010 (registered trademark). The antioxidant may be used in an amount of 0.5 to 2.0 parts by weight per 100 parts by weight of the latex.

The frothing agent mixes the foam latex composition according to the present invention with a gas such as air to froth the composition, and may be a surface active agent. A surface active agent that does not hinder gelation of the latex due to the action of the gelling agent to be discussed later is used. An anionic surface active agent and a nonionic surface active agent may be used. Examples include a potassium salt or a sodium salt of fatty acid and a fatty acid soap of an ammonium salt. Among these, a potassium salt or a sodium salt of ricinoleic acid, oleic acid, lauric acid, and castor oil fatty acid is preferably used.

The frothing agent may be used in an amount of 0.5 to 5.0 parts by weight per 100 parts by weight of the latex. In some cases, a surface active agent is used in the latex and the emulsion in order to stabilize the latex particles and the emulsion particles, and such a surface active agent can also serve as the frothing agent. In this case, the frothing agent may be used in a smaller amount.

The foam stabilizer acts to stabilize foam and prevent the collapse of foam when the gelling agent acts on the frothed latex composition to solidify the composition. The foam stabilizer may be a cationic surface active agent and nitrogen-containing cationic organic matter. Specific examples include Trimene Base (trade name; a product of Addivant USA), an alkyl quaternary ammonium salt compound, a cationic polymer, an amphoteric surface active agent, a betaine surface active agent, a dicyandiamide diethylenetriamine polycondensate, polyethyleneimine, etc.

The foam stabilizer may be used in an amount of 0.1 to 5.0 parts by weight, as the solid content of the foam stabilizer, per 100 parts by weight of the latex or the emulsion. The concentration of the foam stabilizer to be added to the latex may be 10% to 50% as an aqueous solution.

The zinc oxide is combined with the gelling agent to be discussed later to gelatinize the foamed latex composition. The zinc oxide is also combined with the vulcanization accelerator to promote vulcanization. The zinc oxide may be zinc oxide produced by a method such as the French process, the American process, and a wet precipitation process in which zinc chloride is used as a raw material. Zinc oxide in the form of fine particles and containing few impurities is preferably used. Specific examples include Type 1, Type 2, and Type 3 of the JIS Standard (These types are classified in accordance with the grade of the zinc oxide. For the appearance, Type 1 and Type 2 are white in color, and Type 3 is not defined. Type 1 and Type 2 have a purity of 99.5% or more, and Type 3 has a purity of 99.0% or more. Type 1, Type 2, and Type 3 contain lead in a proportion of 0.005% or less, 0.03% or less, and 0.3% or less, respectively). The zinc oxide may be used in an amount of 0.5 parts by weight to 5.0 parts by weight, preferably 1.0 parts by weight to 3.0 parts by weight, per 100 parts by weight of the latex or the emulsion.

The gelling agent solidifies the foamed latex composition, and may be a silicofluoride salt such as sodium silicofluoride and potassium silicofluoride, a persulfate, a peroxide, etc. These substances are dissolved or decomposed to generate acid. Consequently, the pH of the latex composition is lowered, which lowers colloid stability and promotes unification of the latex or emulsion particles to solidify the particles in the frothed state. This change is called “gelation”. The gelling agent may be used in an amount of 0.5 parts by weight to 5.0 parts by weight, preferably 1.0 parts by weight to 4.0 parts by weight, as the solid content per 100 parts by weight of the latex or the emulsion.

While the foam latex composition is produced by combining these chemicals, compounding agents such as an ultraviolet absorber, a softening agent, a filler, a color material, an antibacterial agent, and an antifungal agent may be used, as appropriate, besides the above chemicals.

In the foam molding, the foam latex composition is mixed with a gas such as air to be frothed, and the gelling agent is caused to act on the composition to solidify (gelatinize) the composition, while being maintained in the frothed state, to mold sponge. The frothed latex composition is injected into a die, and thereafter gelatinized to impart a shape. After that, the composition is vulcanized to impart rubber elasticity.

To mix a gas, a wire whip mixer, a Hobart mixer, etc. of a batch type may be used. A pin mixer, an Oakes mixer, etc. of a continuous type may also be used.

The vulcanization is performed by applying heat to the gelatinized latex composition, and imparts elasticity to the latex sponge. The vulcanization may be performed using vapor, a heating medium, high frequencies, etc., and may be performed for 10 minutes to 2 hours at 95 to 150° C.

The resulting latex sponge is cut as necessary, and subjected to cleaning according to the present invention. As a result of the cleaning, a discolorable substance is removed to obtain sponge that is not easily discolored over time.

The cleaning according to the present invention is performed using an oxidizing cleaning liquid. The oxidizing cleaning liquid may be an aqueous solution of a peroxide, a persulfate, a percarbonate, a perborate, a perchlorate, a periodate, etc. Specific examples include hydrogen peroxide, peracetic acid, potassium persulfate, sodium persulfate, ammonium persulfate, potassium percarbonate, sodium percarbonate, sodium perborate, sodium perchlorate, sodium periodate, etc. Among these, sodium percarbonate is particularly preferable, since it does not require pH adjustment and there is little risk in handling it. These chemicals may be adjusted to an appropriate pH. For example, hydrogen peroxide may be alkalized to improve the cleaning effect.

The concentration of the cleaning liquid may be 0.05 to 5.0%, preferably 0.1 to 0.4%. If the concentration is lower, the cleaning takes more time and the cleaning effect is lower. If the concentration is higher, the sponge may be spotted, which is not preferable. The cleaning liquid is used in an amount of 5 parts by weight to 50 parts by weight, preferably 10 parts by weight to 25 parts by weight, per 1 part by mass (dry weight) of the sponge to be cleaned. If the cleaning liquid is used in a smaller amount, the cleaning liquid is not distributed all over the sponge, which may cause partial pinking. If the cleaning liquid is used in a larger amount, white spots may be caused in the sponge, which is not preferable. The cleaning temperature is preferably 50 to 90° C., at which the cleaning liquid is decomposed etc. to be able to demonstrate the cleaning effect. The cleaning time may be 10 to 100 minutes.

The cleaning may be performed using a washing machine. A drum-type washing machine with chemical resistance is preferably used. The cleaning may also be performed by soaking. In that case, the cleaning liquid is preferably stirred from time to time to be distributed uniformly. The cleaning may be performed from a state in which the sponge is dry or from a state in which the sponge is wet. In the case where the cleaning is performed from a state in which the sponge is wet, the cleaning liquid is preferably prepared with the amount of water contained in the sponge subtracted.

After the cleaning, the sponge is rinsed and dried to obtain the sponge according to the present invention.

The sponge according to the present invention produced in this manner is not significantly discolored over time, and can be evaluated by performing an accelerating test as follows.

A transparent glass container that can be tightly sealed is prepared. Test sponge is put in the container. A small amount of ammonia water is put to create an ammonia atmosphere. The test sponge is irradiated with fluorescent light for 24 hours. Specifically, 0.4 ml of 25% ammonia water is dropped (1.33 ml/l, ammonia concentration: 0.03 g/l) into a box body (capacity: about 3 l) with dimensions of 230 mm×160 mm and a depth of 82 mm, a sample is placed in the box, which is lidded with a glass plate and irradiated with a sunlight color fluorescent lamp (30-W circular type) from 35 cm above for 24 hours (illuminance: 2200 lux). When sponge that is not subjected to the cleaning according to the present invention is tested by the above method, the sponge is discolored to a pink to reddish brown color. In the case where the sponge according to the present invention is tested, on the contrary, the sponge is not discolored, and the effect of the cleaning according to the present invention can be appreciated.

With the present invention, the color difference ΔE*ab between before and after the test can have a value of 0 to 10. When the color difference value is 10, the sponge according to the present invention is discolored over time to a hardly noticeable degree, and determined to have practically sufficient resistance to discoloration, compared to the discoloration over time of various kinds of natural rubber latex sponge.

### EXAMPLES—PRODUCTION OF NATURAL RUBBER LATEX SPONGE

### Example 1

Preparation of Foam Composition—Air is blown to natural rubber latex of a high ammonia type (with a solid content of 60% and a pH of 11.0) while being stirred, to volatilize ammonia and lower the pH to 9.8. 2.5 Parts by weight of sulfur as the vulcanizing agent, 2.5 parts by weight of EZ (zinc diethyldithiocarbamate) as the vulcanization accelerator, and 1.0 part by weight of BHT (2,6-di-tertiary butyl-p-cresol) as the antioxidant were added, each as a water dispersion containing an active ingredient of 50%, to 100 parts by weight of the solid content of the natural rubber latex. 1.5 Parts by weight of potassium oleate as the frothing agent and 0.5 parts by weight of Trimene Base (registered trademark) as the foam stabilizer were also added.

Foam Molding—The foam latex composition, air, zinc oxide, and the gelling agent were mixed and stirred using a pin mixer as a stirrer of the continuous type to be frothed and foamed. The foam is injected into a die, and gelatinized by the action of the gelling agent. After that, the foam is vulcanized to be turned into latex sponge with rubber elasticity.

The zinc oxide was added in an amount of 3.0 parts by weight, and sodium silicofluoride as the gelling agent was also added in an amount of 3.0 parts by weight, per 100 parts by weight of the solid rubber content of the foam latex composition. Air was adjusted such that the weight of the foam was 250 g/l (foaming ratio: 4.0 times). A circular column die with a diameter of about 60 mm was used as the die. The flowability of the foam was lost and the foam became solid 120 seconds after the foam was injected into the die.

After that, the solid was vulcanized for 60 minutes at 100° C. using vapor. After that, the sponge was taken out of the die, washed with water, dehydrated, and cut to a thickness of 8 mm and a diameter of 60 mm.

Cleaning Treatment—1 Part by dry weight of the prepared sponge was soaked in 15 parts by weight of a treatment liquid prepared by solving 0.3 parts by weight of sodium percarbonate in 100 parts by weight of water, and the liquid was heated to 60° C. for 30 minutes while stirring. After that, the sponge was rinsed, dehydrated, and dried to obtain the sponge according to the present invention.

### Example 2

Deproteinized natural rubber latex, or Selatex 3821 (registered trademark; with a solid content of 60%) was used in place of the natural rubber latex of a high ammonia type (with a solid content of 60%) which was used in Example 1. The latex was prepared by decomposing proteins contained in natural rubber latex, separating the proteins through centrifugal separation, and refining the latex, and had a total nitrogen content of 0.15% or less. The latex had a pH of 10.0, and was used as it was without performing pH adjustment.

As in Example 1, 2.5 parts by weight of sulfur, 2.5 parts by weight of EZ (zinc diethyldithiocarbamate) as the vulcanization accelerator, 1.0 part by weight of BHT (2,6-di-tertiary butyl-p-cresol), 0.5 parts by weight of potassium oleate, and 0.5 parts by weight of Trimene Base (registered trademark) were added to 100 parts by weight of the solid content of the prepared latex. The latex contained potassium oleate as the stabilizer, and contained potassium oleate as the frothing agent in a small amount compared to the natural rubber latex. The composition was subjected to foam molding, washed with water, cut, and subjected to a cleaning treatment as in Example 1 to obtain the sponge according to the present invention.

### Example 3

Air is blown to natural rubber latex of a high ammonia type according to Example 1 (with a solid content of 60% and a pH of 11.0) while being stirred, to volatilize ammonia and lower the pH to 9.8. 1.5 Parts by weight of potassium oleate was added to 100 parts by weight of the solid content of the natural rubber latex, and the mixture was stirred until the mixture became uniform. 1.0 Part by weight of a 10% aqueous solution of an alkaline protease as the protein decomposition enzyme was added while stirring. After that, the temperature of the solution was raised to 50° C. while stirring the solution, and kept for 30 minutes. After that, the stirring was stopped, and the solution was left to stand for 24 hours until the temperature was brought to room temperature. The proteins in the latex were decomposed. When protein allergens (Hev-b 6.02) which were peculiar to the natural rubber were measured using the ELISA method, 0.025 μg/g was observed, in contrast to 1.43 μg/g observed in the latex before enzyme decomposition.

As in Example 1, 2.5 parts by weight of sulfur, 2.5 parts by weight of EZ (zinc diethyldithiocarbamate) as the vulcanization accelerator, 1.0 part by weight of BHT (2,6-di-tertiary butyl-p-cresol), and 0.5 parts by weight of Trimene Base (registered trademark) were added to 100 parts by weight of the solid content of the raw material natural rubber latex. Potassium oleate had been added at the time of an operation for enzyme decomposition, and was not added at the time of compounding. The composition was subjected to foam molding, washed with water, cut, and subjected to a cleaning treatment as in Example 1 to obtain the sponge according to the present invention.

### Comparative Example 1

Sponge according to Comparative Example 1 was prepared through the steps of producing the sponge according to Example 1 except that the sponge was dried without performing the final cleaning step.

### Comparative Example 2

Sponge according to Comparative Example 2 was prepared through the steps of producing the sponge according to Example 2 except that the sponge was dried without performing the final cleaning step.

### Comparative Example 3

Sponge according to Comparative Example 3 was prepared through the steps of producing the sponge according to Example 3 except that the sponge was dried without performing the final cleaning step.

Raw Material Latex and Evaluation and Conditions of Sponge Produced

Allergens

The amount of allergen proteins (Hey-b 6.02) in the raw material latex was measured using a latex allergen ELISA kit manufactured by Icosagen AS by the method according to ASTM D7427-16 to calculate the amount of allergen proteins per solid content of the latex.

Pinking

A transparent container that can be tightly sealed was prepared. Test sponge is put in the container. A small amount of ammonia water is put to create an ammonia atmosphere. The test sponge is irradiated with fluorescent light for 24 hours. Specifically, 0.4 ml of 25% ammonia water was dropped (1.33 ml/l, ammonia concentration: 0.03 g/1) into a box body (capacity: about 3 l) with dimensions of 230 mm×160 mm and a depth of 82 mm, a sample was placed in the box, which was lidded with a glass plate and irradiated with a sunlight color fluorescent lamp (30-W circular type) from 35 cm above for 24 hours (illuminance: 2200 lux). For the color difference of the sponge between before and after the test, the color difference (ΔE*ab) in the L*a*b* color system prescribed by JIS Z 8730 was measured using a color difference meter CR-300 (manufactured by Konika Minolta).

Discoloration Over Time

The sponge was left to stand at a place with no direct sunlight in an office, and observed three months later.

Cells

Fineness and Uniformity of Cells (Bubbles) in Sponge

The cells were evaluated as follows.

Very good: The cells were small and uniform.

Good: Some cells were rough/tiny.

Poor: The cells were partially sparse.

Apparent Density

The apparent density was obtained by cutting a rectangular parallelepiped from the sponge and dividing the weight by the apparent volume.

Hardness

The value on an Asker F-type durometer (Kobunshi Keiki Co., Ltd.) was used.

Strength, Elongation, and Modulus

The tensile strength, the elongation at break, and the tensile stress (modulus) at 100% elongation were measured using a No. 1 dumbbell specimen in accordance with JIS K 6251.

TABLE 1 below indicates the results for Examples 1 to 3 and Comparative Examples 1 to 3 described above.

Evaluation of Cosmetic Sponge

The sponge prepared in Examples 1 and 2 and Comparative Examples 1 and 2 were sliced to a thickness of 8 mm, and punched into a circular shape with a diameter of 60 mm. The circumferential end portion of the sponge was polished and processed into a round shape to obtain cosmetic sponge. The cosmetic sponge was used to actually apply make-up, and evaluated the usability and the application performance of the cosmetic sponge. The evaluation was performed by five women using a powder foundation that was available in the market.

For the usability, the touch to the skin, the feel for the skin, and the moistness were checked. The evaluation was made as follows in accordance with the number of persons that gave favorable evaluations for these criteria, and indicated in TABLE 1.

Very good: 5 persons

Good: 3 or 4 persons

Fair: 2 or 3 persons

Poor: 0 or 1 person

For the application performance, how the sponge took the foundation, how the foundation was spread over the skin, the ability to cover, and the ability to prevent spots or streaks were checked. The evaluation was made in the same manner as the usability in accordance with the number of persons that gave favorable evaluations for these criteria, and indicated in TABLE 1.

TABLE 1

| | Unit | Ex. 1 | Ex. 2 | Ex. 3 | Com. Ex. 1 | Com. Ex. 2 | Com. Ex. 3 |
|---|---|---|---|---|---|---|---|
| Raw | | Natural | Deproteinized | Enzyme- | Natural | Deproteinized | Enzyme- |
| material | | rubber | natural | decomposed | rubber | natural | decomposed |
| latex | | | rubber | natural | | rubber | natural |
| | | | | rubber | | | rubber |
| Allergens | μg/g | 1.43 | 0.008 | 0.025 | 1.43 | 0.008 | 0.025 |
| Cleaning | | Oxidizing | Oxidizing | Oxidizing | Water | Water | Water |
| Pinking | ΔE*ab | 3.6 | 5.3 | 5.4 | 12 | 15 | 18 |
| Discoloration | Visual | Not | Not | Not | Discolored | Discolored | Discolored |
| over time | | discolored | discolored | discolored | to light red | to red | to red |
| Cells | Visual | Good | Fair | Very good | Good | Fair | Very good |
| Physical | | | | | | | |
| properties | | | | | | | |
| Apparent | Kg/m³ | 150 | 115 | 130 | 155 | 120 | 130 |
| density | | | | | | | |
| Hardness | Degrees | 58 | 50 | 49 | 60 | 51 | 50 |
| Strength | KPa | 150 | 140 | 145 | 150 | 140 | 145 |
| Elongation | % | 360 | 320 | 520 | 365 | 325 | 525 |
| Modulus | KPa | 43 | 40 | 36 | 45 | 40 | 37 |
| Usability | Monitor | Fair | Good | Very good | Fair | Good | Very good |
| Application | Monitor | Fair | Good | Very good | Fair | Good | Very good |
| performance | | | | | | | |

The natural rubber latex sponge according to the present invention can provide sponge that has a uniform and fine cell structure and that is in a light color and is resistant to discoloration over time. Consequently, the degree of freedom in merchandise planning can be increased to finely meet the needs of the customers. In addition, no much attention is required to manage the inventory period in merchandise management.

The deproteinized natural rubber latex sponge according to the present invention can be used with security with no worries about allergies. In addition, the sponge is soft with low modulus, has a good touch to the skin, and is suitable for use as cosmetic items.

Consequently, the sponge can be used in wiping materials, various kinds of rollers, washing tools, application tools, adsorbents, cushions, mattresses, etc. In addition, the sponge is suitable for use where the sponge is used in direct contact with the skin, and suitable for use as cosmetic items such as cosmetic sponge, eye shadow sponge, and cleansing sponge.

Further, the sponge does not contain raw materials that derive from petroleum, has the double effect in reducing the amount of emission of carbon dioxide, and is friendly to the global environment.

Foam molding is performed after proteins are decomposed using an enzyme. Therefore, it is possible to provide sponge with no worries about allergies without increasing the cost, as the latex cleaning and refining steps are omitted.

1. A natural rubber latex sponge which is not easily discolored over time, characterized by comprising foam-molding natural rubber latex, and cleaning the natural rubber latex using an oxidizing cleaning liquid.

2. The natural rubber latex sponge according to claim 1, characterized by being discolored when irradiated with light for 24 hours in an ammonia atmosphere of 0.03 g/l, discoloration of the natural rubber latex sponge causing a color difference of 0 to 10 between before and after the irradiation in terms of a color difference (ΔE*ab) in an L*a*b* color system prescribed by JIS Z 8730 that is, a color difference calculated in accordance with an opponent color space CIE 1976 (L*a*b* color space), abbreviated to CIE Lab, determined by the International Commission on Illumination, and also prescribed by ISO 7724 and ASTM D2244.

3. The natural rubber latex sponge according to claim 1, wherein the natural rubber latex sponge is produced from deproteinized natural rubber latex.

4. The natural rubber latex sponge according to claim 1, wherein the sponge was cleaned using the oxidizing cleaning liquid, wherein the oxidizing cleaning liquid was an aqueous solution.

5. The natural rubber latex sponge according to claim 1, wherein the oxidizing cleaning liquid comprises a member of the group consisting of an aqueous solution of a peroxide, a persulfate, a percarbonate, a perborate, a perchlorate, and a periodate.

6. The natural rubber latex sponge according to claim 1, wherein the sponge comprises deproteinized natural rubber latex and optionally synthetic rubber latex, and the sponge was cleaned using the oxidizing cleaning liquid.

7. The natural rubber latex sponge according to claim 1, made by a process comprising producing the natural rubber latex sponge from natural rubber latex, wherein optionally the natural rubber latex is deproteinized natural rubber latex, and cleaning the sponge using the oxidizing cleaning liquid.

8. The natural rubber latex sponge according to claim 1, made by a process comprising producing the natural rubber latex sponge from deproteinized natural rubber latex, and cleaning the sponge using the oxidizing cleaning liquid.

9. The natural rubber latex sponge according to claim 1, made by a process comprising producing the natural rubber latex sponge from a mixture comprising natural rubber latex and synthetic rubber latex, and cleaning the natural rubber latex sponge using the oxidizing cleaning liquid.

10. The natural rubber latex sponge according to claim 1, made by a process comprising producing the natural rubber latex sponge from a mixture comprising deproteinized natural rubber latex and synthetic rubber latex, and cleaning the natural rubber latex sponge using the oxidizing cleaning liquid.

11. The natural rubber latex sponge according to claim 1, wherein the oxidizing cleaning liquid comprises an aqueous solution comprising any of hydrogen peroxide, peracetic acid, potassium persulfate, sodium persulfate, ammonium persulfate, potassium percarbonate, sodium percarbonate, sodium perborate, sodium perchlorate, and sodium periodate.

12. The natural rubber latex sponge according to claim 1, wherein the oxidizing cleaning liquid comprises an aqueous solution comprising sodium percarbonate, sodium perborate.

13. A process of producing the natural rubber latex sponge according to claim 1, comprising causing a protein decomposition enzyme to react with natural rubber latex, and foam-molding the natural rubber latex with decomposed matter remaining.

14. A process of producing the natural rubber latex sponge according to claim 1, comprising foam-molding natural rubber latex, and cleaning the natural rubber latex using a cleaning liquid of sodium percarbonate.
